(19) United States
(12) Patent Application Publication
(10) Pub. No.: US 2024/0342456 A1
(43) Pub. Date: Oct. 17, 2024

(54) APPLICATION APPARATUS FOR AT LEAST ONE IMPLANT

(71) Applicant: AMW GMBH, Warngau (DE)

(72) Inventors: Dirk BARTH, Gräfelfing (DE); Alexandra FEUERSINGER, Kolbermoor (DE); Hubert KAFFL, Fischbachau (DE); Lukas OLIV, Rottach-Egern (DE); Stefanie RODLER, Valley (DE); Magnus WIEDENBAUER, Miesbach (DE); Alexander HUBNER, Stammbach (DE); Bernd PAECH, Adelsdorf (DE); Frank SCHNABEL, Feilitzsch (DE); Andreas TITTEL, Oettersdorf (DE); Andrea VILLA, Presseck (DE)

(73) Assignee: AMW GMBH, Warngau (DE)

(21) Appl. No.: 18/292,454

(22) PCT Filed: Jul. 19, 2022
(86) PCT No.: PCT/EP2022/070232

§ 371 Date: Jan. 26, 2024

(30) Foreign Application Priority Data
Aug. 3, 2021 (DE) 10 2021 120 149.7

(51) Int. Cl. A61M 37/00; A61D 19/00

(52) U.S. Cl. CPC A61M 37/0069; A61D 19/00

(57) ABSTRACT

An application apparatus (1) for at least one implant comprises a cannula (3) via which the implant is supplied to a target implantation location. An applicator housing body (32) comprises a depot volume for storing the implant. A plunger (11) of a plunger device (10) can be displaced between an initial position and an implantation position. The plunger device (10) comprises a plunger actuation body (12) to which the plunger (11) is rigidly connected. The applicator housing body (32) is intended here for guiding a displacement movement of the plunger device (10). An initial-position locking device is intended to releasably lock the applicator housing body (32) with the plunger device (10) in the initial position thereof. The initial-position locking device can be switched between a locking position and a functional position in which the plunger device (10) can be displaced in order to displace the plunger (11) relative to the applicator housing body (32). This results in an application apparatus (1), the functional safety of which is improved while having a design which is simultaneously simple and mass-producible.

The invention relates to an application apparatus for at least one implant.

Such an application apparatus is known from EP 3 456 374 B1.

It is an object of the present invention to further develop an application apparatus for at least one implant such that its functional safety is improved while having a design which is simultaneously simple and mass-producible.

This problem is solved according to the invention by an application apparatus having the features indicated in claim 1.

An application apparatus according to the invention for at least one implant comprises a cannula, via which the implant is supplied to a target implantation location, an applicator housing body comprising a depot volume, and a plunger device comprising a plunger.

The “depot volume” is a firmly defined volume region within the application apparatus, provided for storing the implant, which preferably situated in a region of the applicator housing body before the cannula. The depot volume could, however, basically also additionally comprise a region in the cannula, e.g. a rear end of the cannula at or in the applicator housing body.

The plunger can be displaced between an “initial position”, in which a plunger ejection end of the plunger lies on a side of the depot volume facing away from a free cannula tip, and an “implantation position”, in which a plunger ejection end, i.e. a face side of the plunger pointing toward the implant, lies in the region of a free cannula tip. In the initial position, an implant which is to be implanted can therefore still be situated in the provided depot volume, as the plunger is not yet projecting into the depot volume.

The fact that the plunger ejection end is situated “in the region of a free cannula tip”, on the other hand, is to be understood to mean that the plunger ejection end is displaced at least so far forward into the cannula that the implant is positioned in the cannula (preferably at a short distance behind the cannula tip, wherein the implant can then already be situated at the target implantation location when the cannula is inserted correctly into the tissue, but is still surrounded by the cannula. When the cannula is subsequently withdrawn again from the tissue, provision can be made, e.g. through suitable further displacement of the plunger relative to the cannula, that the implant remains at the target implantation location in the tissue, as will be further explained more closely later.

For this, the plunger device has a plunger actuation body to which the plunger is connected. The connection between plunger and plunger actuation body can be configured to be detachable, i.e. the plunger is, for example, screwed in or detachably clamped in the plunger actuation body. A rigid connection is, however, preferably concerned. The applicator housing body itself is configured for guiding a displacement movement of the plunger device between the initial position and the implantation position.

According to the invention, the application apparatus has an initial-position locking device for releasably locking the applicator housing body with the plunger device in its initial position. This initial-position locking device can be switched between a locking position, in which the plunger device is fixed at the applicator housing body in the initial position, and a functional position, in which the plunger device can be displaced in order to displace the plunger relative to the applicator housing body.

By means of the initial-position locking device it is achieved in a functionally safe manner that the application apparatus is not triggered in an unsolicited manner, that therefore the plunger device is displaced in an unsolicited manner from the initial position in the direction of the implantation position. The initial-position locking device can be configured as a detent device with at least one detent nose and/or with at least one detent opening and/or with at least one detent hook.

The initial-position locking device can comprise in particular two different types of detent noses which interact with different counter-bodies. Components of the locking device can be embodied, on the one hand, at the plunger device, in particular at a plunger actuation body, and on the other hand at an applicator housing body.

Main components of the application apparatus can be embodied as plastic components and in particular as injection moulded components. All the components of the application apparatus can be embodied as plastic components. Preferably, however, the cannula and the plunger are made of stainless steel.

The plunger can preferably be rigidly connected to the plunger actuation body.

The present invention relates furthermore to the human-medical, veterinary-medical or cosmetic use of the application apparatus according to the invention.

The use of the application apparatus according to the invention preferably takes place for therapeutical application, particularly preferably for the administering of medicaments, in particular for the administering of (synthetic) analogues of the neurohormone gonadotropin-releasing hormone (GnRH), in particular for the treatment of prostate or breast cancer, endometriosis, uterine myoma, endometrium ablation, precocious puberty (Pubertas praecox), transsexuality, cluster headache and in human-medical and veterinary-medical reproductive medicine.

Further, particularly advantageous embodiments and further developments of the invention will also emerge from the dependent claims and from the following description, wherein the independent claims of a claim category can also be further developed in an analogous manner to the dependent claims and example embodiments of another claim category, and in particular also individual features of various example embodiments or respectively variants can be combined to new example embodiments or respectively variants.

The initial-position locking device is preferably embodied such that its switching between the locking position and the functional position takes place by twisting the plunger device relative to the applicator housing body about a plunger longitudinal axis. Such a locking device can be operated easily and, in particular, intuitively.

The initial-position locking device preferably has a reverse rotation protection, which prevents a reverse displacement of the initial-position locking device from the functional position into the locking position. Such a reverse rotation protection leads to a tamper-proofing of the application apparatus.

The initial-position locking device is preferably configured such that, on a switching of the initial-position locking device from the locking position into the functional position, e.g. compulsorily, an acoustic signal is emitted. For example, through a suitable detent means or suchlike, it can be ensured that, on switching, a distinctly audible clicking takes place.

Basically, the applicator housing body could be manufactured in one part. Preferably, however, the applicator housing body is embodied having multiple parts, in particular two parts, particularly preferably with an implant depot body on the cannula side, i.e. coupled with the cannula, and with a guide body. Preferably, the depot volume for storing the implant is situated here in the implant depot body. The guide body can serve for guiding the displacement movement of the plunger device between the initial position and the implantation position, i.e. the plunger actuation body is mounted in the guide body in a displaceable and guided manner.

The implant depot body is preferably coupled at its rearward end, i.e. at the end pointing away from the cannula, with the plunger actuation body by means of detent connections.

A multi-part or respectively two-part configuration has the advantage that the individual parts are easier to manufacture, for example in an injection moulding method. In addition, an easier introduction of the implant into the depot volume is enabled hereby, for example by the implant being inserted into the implant depot body from the rear before the coupling of the implant depot body with the plunger actuation body.

As is further mentioned later, two separate applicator assemblies can thus be delivered to the implant manufacturer, and the implant manufacturer can provide for the filling with the implant and the final mounting in a simple manner.

In order to ensure a correct mounting of the individual parts, the parts which are to be mounted together can be provided respectively with fitting elements, such as for example grooves and/or webs or suchlike, so that the parts only fit together in precisely one alignment.

In the initial position of the plunger device, the cannula tip can preferably be covered by a protective cap, in order to protect an operator from stab injuries by the cannula as long as the application apparatus is not being used.

The protective cap can additionally have a supporting function in the securing of the plunger device in the initial position, when the protective cap is arranged so that the plunger device can not then be displaced forwards, i.e in the direction of the cannula.

The protective cap is expediently detachably connected to the applicator housing body, preferably with an implant depot body of the applicator housing body. Preferably, the detaching of the protective cap from the applicator housing body takes place by twisting the protective cap relative to the applicator housing body or respectively to the implant depot body about a plunger longitudinal axis. This leads inter alia to a simple design of the application apparatus.

The application apparatus is preferably constructed here so that a first rotation direction in which the protective cap is to be twisted relative to the applicator housing body about the plunger longitudinal axis, in order to detach the protective cap from the applicator housing body, is aligned to a second rotation direction, in which the applicator housing body is to be twisted relative to the plunger device about the plunger longitudinal axis, in order to switch the initial-position locking device from the locking position into the functional position.

Through this construction, the risk can be reduced that the user erroneously releases the lock instead of unscrewing the protective cap when, by mistake, he wants to unscrew the protective cap in the wrong direction, and in so doing holds the application apparatus behind at the plunger device and twists the protective cap relative thereto. When the protective cap allows itself to be detached more easily with the correct rotation direction than the twisting is possible from the locking position into the functional position, it is ensured that the protective cap is unscrewed first. In the case of a wrong rotation direction, the risk would otherwise exist that the applicator housing body is co-rotated with the protective cap relative to the plunger device and thus an unlocking takes place unintentionally.

The application apparatus preferably has a needle guard body which, in the initial position of the plunger device, is arranged in a “neutral position” in which the cannula projects (through an anterior face side of the needle guard body, pointing away from the plunger actuation body, which end face can have a central opening through which the cannula passes in the neutral position). In this “neutral position”, the needle guard body has no protective effect, as the cannula tip is not covered by the needle guard body.

In the neutral position of the needle guard body, the latter can preferably be situated at the height of the implant depot body, i.e. in the initial position, the needle guard body can be arranged in the implant depot body.

The needle guard body is configured and mounted on or respectively in the applicator housing body so that, on displacement of the plunger device, it is transferred from the initial position further, beyond the implantation position, into a “protective position”, in which the needle guard body receives the free cannula tip. This means that the plunger device can be configured so that it is shifted out from the implantation position further forwards, to the cannula tip, and in so doing entrains the needle guard body, so that the anterior face side of the needle guard body is then displaced in front of the cannula tip and the cannula tip is consequently situated in the needle guard body.

A needle guard body has proved to be successful for protection of the user from stab injuries. By means of the needle guard body, in addition, a “retraction force” can be exerted onto the cannula for removing the latter from the target implantation location. In fact, the needle guard body is supported with its anterior face side as abutment against the surface of the tissue in which the implant is to be deposited, e.g. the skin of an implant recipient, and the needle guard body is then shifted further forwards relative to the cannula by means of the plunger device. That equates to a retraction of the cannula into the needle guard body and thus out from the tissue against which the needle guard body lies. Through the parallel movement of needle guard body and plunger, the implant thereby remains automatically at the target implantation location which has already been reached in the implantation position.

The application apparatus is preferably configured such that the plunger ejection end, in the protective position of the needle guard body, lies at a defined deposition distance in front of the anterior face side of the needle guard body. The deposition distance is preferably at least 4.0 mm, particularly preferably at least 4.5 mm.

The deposition distance determines ultimately the depth of the target implantation location under the tissue surface or respectively skin surface (therefore the shortest distance between deposited implant and surface), when the cannula was inserted correctly up to the stop of the needle guard body, and the plunger and the needle guard body are pushed forwards, which corresponds to a retraction of the needle out from the body.

The deposition distance can be adjusted through the appropriate choice of the lengths or respectively length ratios of the corresponding parts of the application apparatus, such as in particular the plunger rod, of the plunger actuation body and of the needle guard body etc., in the desired manner.

The application apparatus preferably has a protective-position locking device for locking the plunger device on the applicator housing body, preferably on the implant depot body, after reaching the protective position.

Such a protective-position locking device can prevent the plunger device from leaving the protective position undesirably, once the protective position has been reached, and thus provides for a secure function of the application apparatus, and in particular for a secure effect of the needle guard body.

As already mentioned, the application apparatus can preferably have at least two applicator assemblies which are initially separate and are preferably able to be connected to one another via a detent connection.

Preferably here, one of the two applicator assemblies can be embodied as depot assembly and can comprise the cannula and the implant depot body. The other of the two applicator assemblies, on the other hand, can preferably be embodied as plunger assembly and can comprise the plunger device and the guide body.

Such a configuration of the application apparatus makes it possible to produce the two assemblies initially separately from one another and to carry out final assembly after insertion of the implant.

Basically, however, a separation in assemblies could also occur at a different place. For example, the implant depot body or the plunger assembly itself could be configured in two parts.

Particularly preferably here the two assemblies could be configured so that they can be mounted exclusively in a directed manner and not rotated inadvertently through 180º.

This is able to be realized e.g. preferably with the aid of a groove on one of the assemblies and a web, fitting thereto, on the other assembly.

Other connections of the applicator assemblies are also possible, e.g. by means of an adhesive connection.

The depot assembly can have, in addition, the already mentioned protective cap and/or the needle guard body. These configurations of the depot assembly have proved to be successful for achieving an efficient mountability.

An example embodiment of the invention is explained more closely in the following with the aid of the drawings. These show:

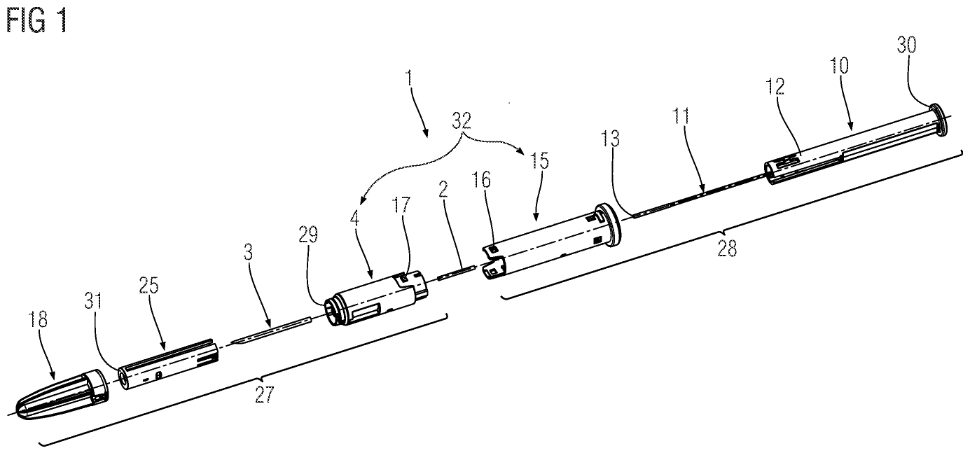

FIG. 1 an exploded view of an application apparatus for at least one implant;

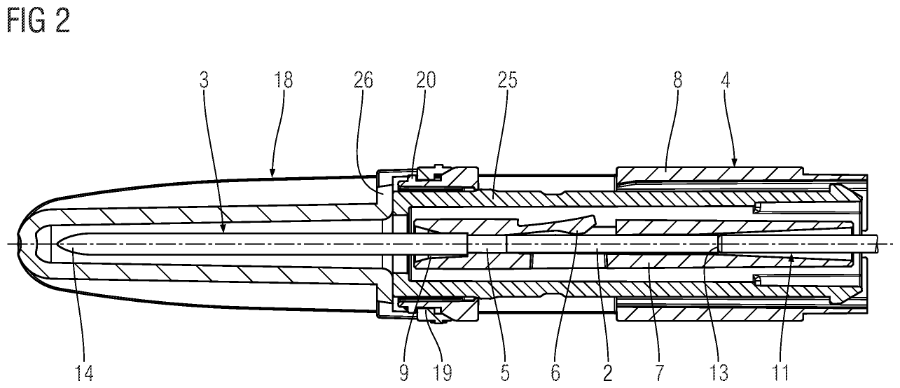

FIG. 2 an axial section through a portion, on the cannula side, of the finished mounted application apparatus in an initial position;

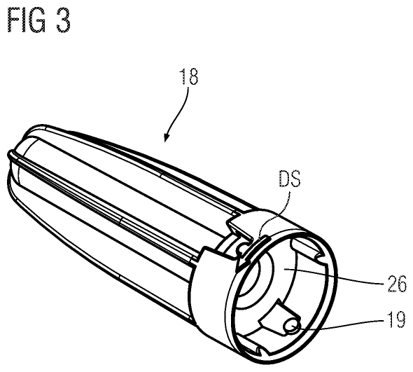

FIG. 3 in perspective, a protective cap of the application apparatus;

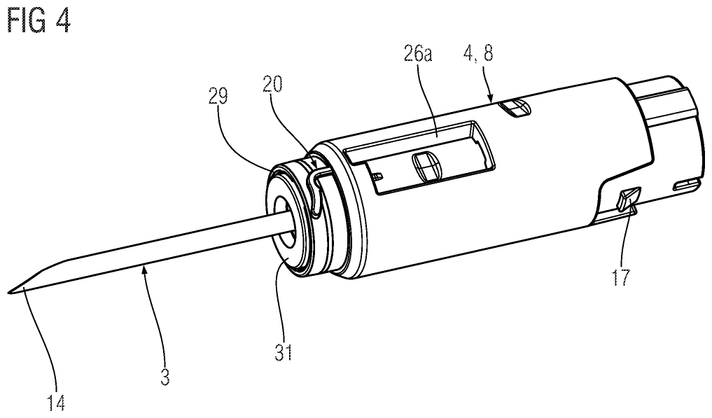

FIG. 4 in perspective, a cannula and a portion, on the cannula side, of an implant depot body of the application apparatus;

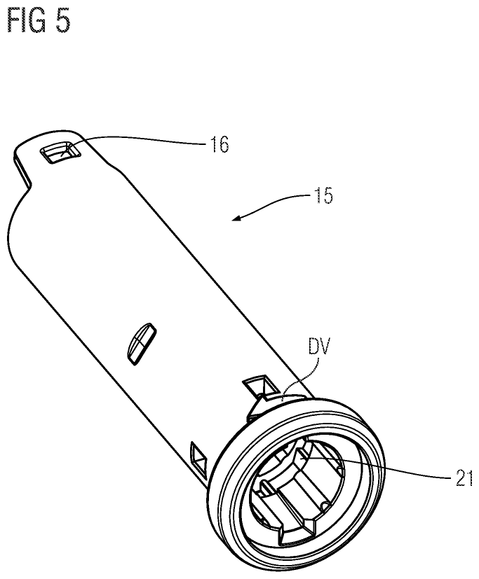

FIG. 5 in perspective, an applicator housing body of the application apparatus;

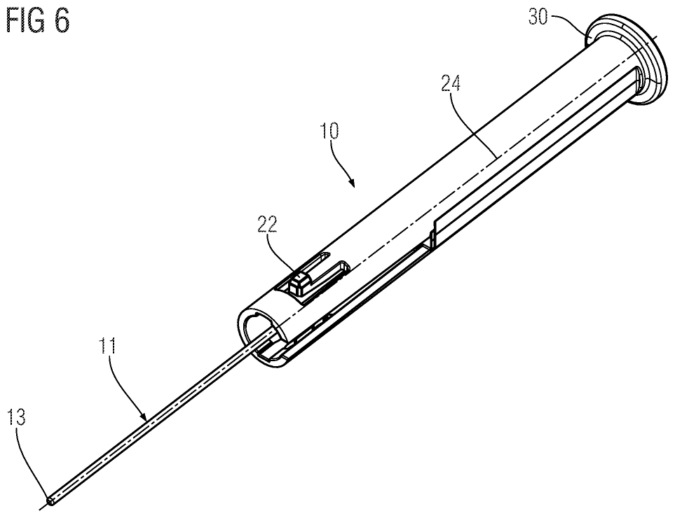

FIG. 6 a plunger device of the application apparatus with a plunger and with a plunger actuation body, viewed from a first viewing direction;

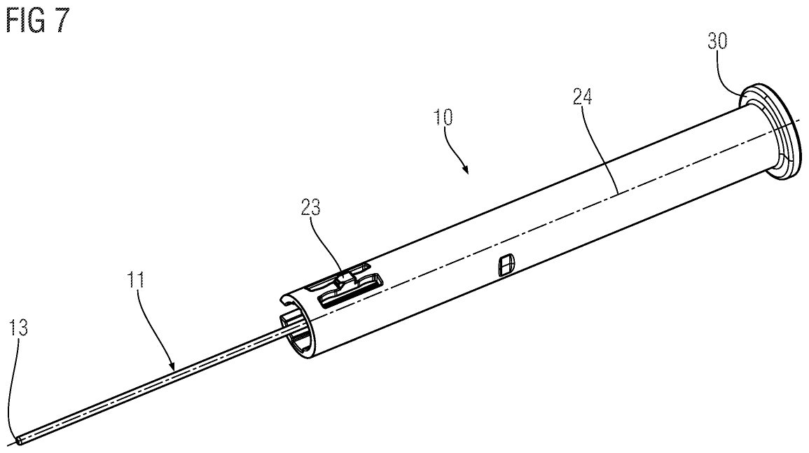

FIG. 7 the plunger device according to FIG. 6, viewed from a second viewing direction, in which the plunger device is twisted approximately about 90° about a plunger longitudinal axis, compared to FIG. 6;

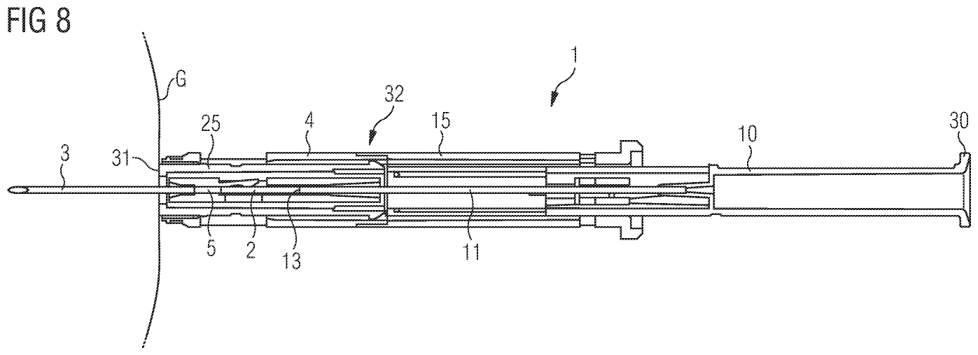

FIG. 8 a longitudinal section through the application apparatus according to FIG. 1 in an initial position of the plunger device, wherein the cannula of the application apparatus is already inserted into the tissue for the application of the implant;

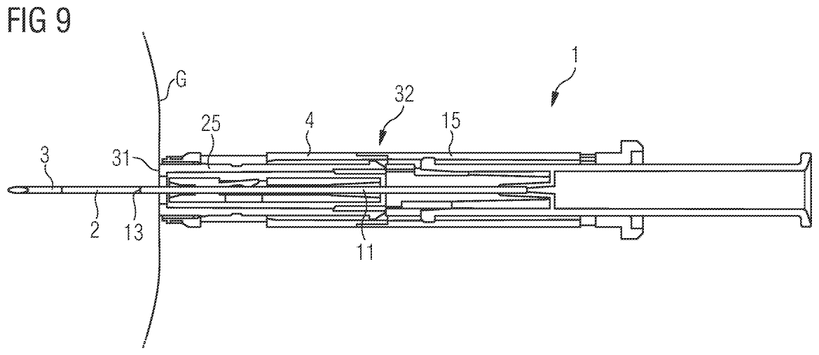

FIG. 9 a longitudinal section through the application apparatus as in FIG. 8, but in the implantation position, wherein the plunger actuation body abuts the needle guard body;

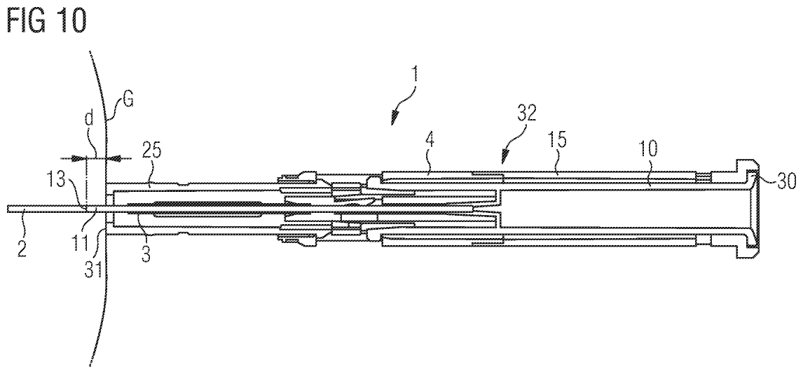

FIG. 10 a longitudinal section through the application apparatus as in FIG. 8 or 9, but in the protective position of the needle guard body, with plunger pushed further forward, so that the plunger ejection end lies outside the cannula tip and still in the tissue;

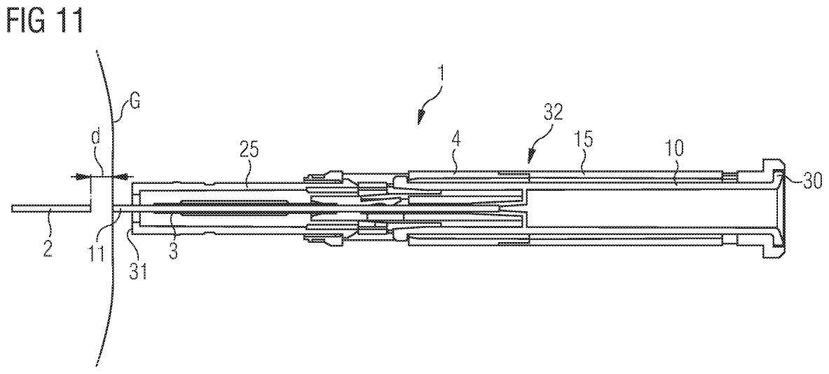

FIG. 11 a longitudinal section through the application apparatus as in FIG. 10, wherein the plunger is withdrawn from the tissue.

An application apparatus 1 serves for the implantation of at least one implant 2 (cf. FIGS. 1 and 2).

Main components of the application apparatus 1 are shown in the exploded view of FIG. 1.

The application apparatus 1 has a cannula 3, via which the implant 2 is supplied to a target implantation location, for example in the subcutaneous abdominal tissue of a patient. The cannula 3 is made of stainless steel. The plastic components of the application apparatus 1 can be manufactured as injection moulded components.

The application apparatus 1 has, furthermore, an implant depot body 4 with a depot volume 5 (cf. FIG. 2) for storing the implant 2. The implant depot body 4 is made of plastic material and can be manufactured for example from methyl methacrylate butadiene styrene (MBS). For fixing the implant 2 in the depot volume 5, the implant depot body 4 has a fixing element 6 in the form of a resilient nose. This nose 6 makes provision, when the implant 2 has reached a target position in the depot volume 5, for a friction-fitting and/or form-fitting fixing of the implant 2 in the depot volume 5. Alternatively or additionally, the fixing element 6 can bring about a clamping of the implant 2.

The depot volume 5 is formed by an inner sleeve portion 7 of the implant depot volume 4. This inner sleeve portion 7 is connected to an outer sleeve portion 8 of the implant depot body 4 via a radially running web which can not be seen more closely in the drawing. The two sleeve portions 7, 8 are respectively one-piece, integral components of the implant depot body 4, which can be produced by injection moulding.

On the cannula side, the inner sleeve portion 7 has a conically tapering cannula receiving region 9 for holding an end portion of the cannula 3 on the implant side. The cannula 3 is fixed, preferably glued or pressed, on the inner sleeve portion 7 via the cannula receiving region 9. The depot volume 5 is aligned with a lumen of the cannula 3.

The application apparatus 1 has, furthermore, a plunger device 10 with a plunger 11 and with a plunger actuation body 12. The plunger actuation body 12 is rigidly connected to the plunger 11. In this case, the plunger 11 is manufactured from stainless steel. The plunger actuation body 12 is made of plastic, for example of polybutylene terephthalate (PBT).

The plunger 11 has an external diameter which is adapted to an internal diameter on the one hand of the depot volume 5 and on the other hand of the lumen of the cannula 3. The entire plunger device 10, and thus the plunger 11, can be displaced between an initial position and an implantation position. In the initial position of the plunger 11, the depot volume 5 lies between the plunger 11 and the end portion of the cannula 3 (see also FIG. 8). In the implantation position, a plunger ejection end 13 lies in the region of a free cannula tip 14 of the cannula 3, wherein the plunger ejection end 13 is displaced at least so far forward into the cannula 3 that the implant is positioned in the cannula, and namely already at the target implantation location (but still surrounded by the cannula 3), when the cannula 3 is inserted correctly into the tissue G (see FIG. 9).

The application apparatus 1 comprises furthermore an applicator housing body 32 with a guide body 15 for guiding a displacement movement of the plunger device 10, and in particular of the plunger actuation body 12, between the initial position and the implantation position of the plunger device 10. The applicator housing body 32, and in particular the guide body 15, is preferably manufactured from plastic, in particular from MBS.

The guide body 15 is rigidly, and in particular non-detachably, connected together with the implant depot body 4 with formation of the applicator housing body 32, via a detent connection with detent openings 16 in the guide body 15 and detent hooks 17 on the implant depot body 4, when application apparatus 1 is completely mounted. This connection of the guide body 15 with the implant depot body 4 is carried out in a torsion-proof manner via groove and webs, so that in particular an incorrect mounting of these two components (e.g. twisted through) 180° with one another is prevented. Corresponding grooves and webs are provided in a sleeve profiling of the implant depot body 4 on the one hand and of the guide body 15 on the other hand, for the anti-torsion protection of these two components with respect to one another.

Furthermore, the application apparatus 1 has a protective cap 18 for covering of the cannula tip 14 and of a further exposed portion of the cannula 3 in the initial position of the plunger device 10. The protective cap 18 is detachably connected to the implant depot body 4 via a detent connection, which is configured in the manner of a bayonet connection, wherein the protective cap 18 is twisted relative to the implant depot body 4 about the plunger longitudinal axis 24 in a first rotation direction DS (see FIGS. 3 and 6). For this, the protective cap has detent noses 19, which interact with a bayonet connecting link 20, which is embodied externally in the outer sleeve portion of the implant depot body 4 in a region thereof on the cannula side.

The protective cap 18 is made from plastic, in particular from polypropylene (PP).

The application apparatus 1 has, furthermore, an initial-position locking device 21 to 23 to releasably lock the applicator housing body 32, here the guide body 15, with the plunger device 10 in the initial position thereof. This initial-position locking device has locking units on the housing body side in the form of detent hooks 21, which are arranged internally in the sleeve-shaped applicator housing body 15 (cf. FIG. 5). Furthermore, the initial-position locking-position device has locking elements on the plunger side in the form of a first type of detent nose 22 (cf. FIG. 6) and of a second type of detent nose 23 (cf. FIG. 7).

The initial-position locking device 21 to 23 can be switched between a locking position, in which the plunger device 10 is fixed on the applicator housing body 32 or respectively on the guide body 15 in the initial position, and a functional position, in which the plunger device 10 can be displaced for displacing the plunger 11 relative to the applicator housing body 32 or respectively guide body 15. This switching of the initial-position locking device 21 to 23 between the locking position and the functional position takes place by twisting of the plunger device 10 relative to the applicator housing body 15 about a plunger longitudinal axis 24 (cf. e.g. FIG. 6) into a second rotation direction DV (see FIG. 5). These two positions are illustrated in FIGS. 6 and 7. The detent hook 21 in the applicator housing body 15 serves furthermore as reverse rotation protection of the initial-position locking device 21 to 23. This reverse rotation protection 21 prevents a reverse displacing of the initial-position locking device 21 to 23 from the functional position back into the locking position.

The detent nose 23 of the second type, which engages into a corresponding groove in the applicator housing body 32 or respectively guide body 15, constitutes a part of the locking through the initial-position locking device 21 to 23 in the locking position. In the functional position, the detent noses 23 of type two engage behind the detent hooks 21, which prevent a reverse rotation into the locking position.

Furthermore, the application apparatus 1 has a needle guard body 25. The latter is arranged in the initial position of the plunger device 10 in a neutral position at the height of the implant depot body 4 (cf. FIG. 2). The needle guard body 25 has a sleeve basic shape and is arranged in the neutral position between the inner sleeve portion 7 and the outer sleeve portion 8 of the implant depot body 4. The connecting web between the two sleeve portions 7, 8 penetrates a corresponding longitudinal slot in the needle guard body 25, which is not shown in the drawing, and permits an axial displacement of the needle guard body 25 relative to the implant depot body 4. With the protective cap 18 put in place, the needle guard body 25 is secured in the neutral position via abutment against a securing collar 26 of the protective cap 18.

The needle guard body 25 is manufactured from plastic material, in particular from PBT.

On displacement of the plunger device 10 in the direction of the cannula tip 14 beyond the implantation position, the needle guard body 25 is entrained by the plunger actuation body 12 and transferred into a protective position (see FIGS. 10 and 11). In this protective position, the needle guard body 25 is displaced up to the free cannula tip 14 of the cannula 3 (i.e. the anterior face side 31 of the needle guard body lies in front of the cannula tip 14) and thus covers the cannula 3 for protection from injuries, in particular at the cannula tip 14. In this position, as illustrated, the plunger 11 can also project forward out from the cannula 3.

Furthermore, the application apparatus 1 has a protective-position locking device for locking the plunger device 10 on the implant depot body 4 when the needle guard body 25 has reached its protective position. Part of this protective position locking device can be the detent nose 22 of the first type, already mentioned above (cf. FIG. 6). Furthermore, a detent window 26a (cf. FIG. 4), which is embodied in the outer sleeve portion 8 of the implant depot body 4, belongs to this protective-position locking device.

Before its final mounting, the application apparatus 1 has two applicator assemblies which are initially separate and are able to be connected to one another via the detent connection 16, 17, namely a depot assembly 27 and a plunger assembly 28 (cf. FIG. 1). The depot assembly 27 comprises the cannula 3 and the implant depot body 4. Furthermore, the depot assembly 27 comprises the protective cap 18 and the needle guard body 25. The plunger assembly 28 comprises the plunger device 10 and the guide body 15.

Before the final mounting of the application apparatus 1, the active substance implant 2 is inserted into the depot volume 5. The two assemblies 27, 28 are subsequently put together at the final mounting and are then rigidly connected to one another via the detent connection 16, 17. The finished mounted application apparatus 1 could then be sterilised, for example, and sealed into a sterile packaging. Likewise, the entire system (application apparatus with packaging bag) could be finally irradiated for sterilisation, for example with gamma- or X ray irradiation. Theoretically, the application apparatus can, however, also be used for non-sterile purposes.

Depending on the execution of the application apparatus 1, various sizes, in particular various diameters of the depot volume 5 can be provided for receiving implants 2 of different sizes. Thus, various implant sizes can be administered with basically identically constructed application apparatuses in the manner of the application apparatus 1.

In the initial position, in addition on the one hand the plunger actuation body 12 lies axially against the needle guard body 25, and the latter in turn lies axially against a securing collar 26 of the protective cap 18, so that the protective cap 18 prevents a triggering of the application apparatus 1, therefore a transferring of the plunger device 10 from the initial position into the implantation position (cf. FIGS. 1 and 2).

In the use of the application apparatus 1, firstly the protective cap 18 is detached from the implant depot body 4 by overcoming the bayonet connection 19, 20. Subsequently, the plunger actuation body 12, and thus the entire plunger device 10, is twisted from the locking position into the functional position of the initial-position locking device 21 to 23.

In order to prevent that, on an attempt to detach the protective cap in the wrong direction, by mistake the initial-position locking device 21 to 23 is released, the first rotation direction DS (see FIG. 3), in which the protective cap 18 is to be twisted for detaching relative to the implant depot body 4 about the plunger longitudinal axis 24, is aligned to the second rotation direction DV (see FIG. 5), in which the applicator housing body 15 is to be twisted relative to the plunger device 10, in order to switch the initial-position locking device 21 to 23 from the locking position into the functional position.

On use of the applicator, the cannula 3 is placed up to the stop, formed by a face side 29 of the implant depot body 4 and/or the anterior face side 31 of the needle guard body 25, under the skin of the patient G (see FIG. 8).

An actuation end 30 of the plunger actuation body 12 is now actuated for example by the thumb of a user. The plunger device 10 is thereby pushed through the applicator housing body 15 in the direction of the cannula 3 into the implantation position. In so doing, the plunger 11 pushes the implant 2 out from the depot volume 5 through the cannula 3 into a position which corresponds to the desired target implantation location, wherein, however, the implant 2 is still surrounded by the cannula 3. This implantation position is shown in FIG. 9.

In the implantation position, the plunger actuation body 12 abuts with its anterior end on the rear end of the needle guard body 25, so that then on a further shifting of the plunger device 10 forwards, i.e. beyond the implantation position, the needle guard body 25 is pushed parallel to the plunger 11 forward via the cannula 3. An anterior face side 31 undertakes here the stop function at the skin G of the patient around the cannula insertion site. In this way, a type of retraction takes place of the cannula 3 out of the skin G into the needle guard body, wherein, at the same time, it is ensured by means of the plunger 11 that the implant 2 remains substantially at the target implantation location under the skin G. This is shown in FIG. 10.

Potentially, at the start of the withdrawal of the cannula 3, the implant 2 can still be pushed forward slightly within the cannula 3 (so that the target implantation location can still move a little), depending on how intensively the anterior face side 31 of the needle guard body 25 can still be pressed into the tissue G. As can be readily seen from FIG. 10, ultimately the deposition distance d between the plunger ejection end 13 and the anterior face side 31 of the needle guard body 25 in the protective position determines the depth of the target implantation location under the skin surface.

In this protective position of the needle guard body 25 and of the plunger device 10, the plunger device 10 engages here via the protective-position locking device at the implant depot body 4, so that preferably a returning of the plunger device 10 out from the protective position back into the initial position is no longer possible. In the protective position, the needle guard body 25 is pushed entirely over the cannula 3 and protects the user from injuries, wherein, however, the plunger 11 already projects at the front out from the cannula 3.

The application is completed when the cannula 3 is removed entirely from the skin G and, in so doing, the plunger 11 has pushed the implant 2 entirely out from the cannula 3, and also the plunger 11 was drawn out from the skin G. This is shown in FIG. 11. The implant then remains securely at the target implantation location, substantially at the deposition distance d, under the skin surface.

The application of just one implant by the application apparatus was described above. Basically, however, it is also possible to embody the application apparatus 1 so that several implants can be applied by this, e.g. in a type of “chain” of several (preferably shorter) implants in succession.

1. An application apparatus (1) for at least one implant (2)
comprising a cannula (3), via which the implant (2) is supplied to a target implantation location,
comprising an applicator housing body (32) with a depot volume (5) for storing the implant (2),
comprising a plunger device (10) with a plunger (11), which can be displaced between
an initial position, in which a plunger ejection end (13) of the plunger (11) lies on a side of the depot volume (5) facing away from a free cannula tip (14), and
an implantation position, in which a plunger ejection end (13) lies in the region of a free cannula tip (14),
wherein the plunger device (10) comprises a plunger actuation body (12) to which the plunger (11) is connected, preferably in a rigid manner,
wherein the applicator housing body (32) is configured for guiding a displacement movement of the plunger device (10) between the initial position and the implantation position,
comprising an initial-position locking device (21 to 23) to releasably lock the applicator housing body (32) with the plunger device (10) in the initial position thereof, wherein the initial-position locking device (21 to 23) can be switched between
a locking position, in which the plunger device (10) is fixed on the applicator housing body (32) in the initial position, and
a functional position, in which the plunger device (10) can be displaced in order to displace the plunger (10) relative to the applicator housing body (32).

2. The application apparatus according to claim 1, characterized in that the initial-position locking device (21 to 23) is configured such that the switching thereof between the locking position and the functional position takes place by twisting of the plunger device (10) relative to the applicator housing body (32) about a plunger longitudinal axis (24).

3. The application apparatus according to claim 1, characterized in that the initial-position locking device (21 to 23) comprises a reverse rotation protection (21) which prevents a reverse displacement of the initial-position locking device (21 to 23) from the functional position into the locking position.

4. The application apparatus according to claim 1, characterized in that the initial-position locking device (21 to 23) is configured such that an acoustic signal is emitted on a switching of the initial-position locking device (21 to 23) from the locking position into the functional position.

5. The application apparatus according to claim 1, characterized in that the applicator housing body (32) is configured having two parts, with an implant depot body (4) on the cannula side, in which preferably the depot volume (5) is situated for storing the implant (2), and with a guide body (15) for guiding the displacement movement of the plunger device (10) between the initial position and the implantation position.

6. The application apparatus according to claim 1, characterized by a protective cap (18) for covering the cannula tip (14) in the initial position of the plunger device (10).

7. The application apparatus according to claim 6, characterized in that the protective cap (18) is detachably connected to the applicator housing body (32), so that preferably a detaching of the protective cap (18) from the applicator housing body (32) takes place by twisting the protective cap (18) relative to the applicator housing body (32) about a plunger longitudinal axis (24).

8. The application apparatus according to claim 7, characterized in that a first rotation direction (DS), in which the protective cap (18) is to be twisted relative to the applicator housing body (32) about the plunger longitudinal axis (24), in order to detach the protective cap (18) from the applicator housing body (32), is aligned to a second rotation direction (DV), in which the applicator housing body (32) is to be twisted relative to the plunger device (10) about the plunger longitudinal axis (24), in order to switch the initial-position locking device (21 to 23) from the locking position into the functional position.

9. The application apparatus according to claim 1, characterized by a needle guard body (25) which, in the initial position of the plunger device (10), is arranged in a neutral position in which the cannula (3) projects out from the needle guard body (25), and which, on the displacement of the plunger device (10) from the initial position is transferred beyond the implantation position into a protective position, in which the needle guard body (25) receives the free cannula tip (14).

10. The application apparatus according to claim 9, characterized in that the application apparatus is configured such that the plunger ejection end (13) in the protective position of the needle guard body (25) lies at a defined deposition distance (d) in front of an anterior face side (31) of the needle guard body (25).

11. The application apparatus according to claim 10, characterized in that the deposition distance (d) is at least 4.0 mm, preferably at least 4.5 mm.

12. The application apparatus according to claim 9, characterized by a protective position locking device for locking the plunger device (10) on the applicator body (32) after reaching the protective position.

13. The application apparatus according to claim 1, characterized by two applicator assemblies (27, 28) which are initially separate and are able to be connected to one another via a detent connection (16, 17),
wherein preferably one of the two applicator assemblies is configured as depot assembly (27) and comprises the cannula (3) and the implant depot body (4),
wherein preferably the other of the two applicator assemblies is configured as plunger assembly (28) and comprises the plunger device (10) and the guide body (15).

14. The application apparatus according to claim 13, characterized in that the depot assembly (27) comprises the protective cap (18) and/or the needle guard body (25).

15. The application apparatus according to claim 1 for human-medical, veterinary-medical or cosmetic use.

16. An application apparatus for use in the administering of medicaments, preferably of (synthetic) analogues of the neurohormone gonadotropin-releasing hormone (GnRH), particularly preferably for the treatment of prostate or breast cancer, endometriosis, uterine myoma, endometrium ablation, precocious puberty (Pubertas praecox), transsexuality, cluster headache and in human-medical and veterinary-medical reproductive medicine.
